(19) United States
(12) Patent Application Publication
(10) Pub. No.: US 2020/0011852 A1
(43) Pub. Date: Jan. 9, 2020

(54) METHODS FOR PREDICTING THE ANTICANCER THERAPEUTIC RESPONSE OF L-ASCORBIC ACID

(71) Applicant: Chang Hwan YEOM, Seoul (KR)

(72) Inventors: Chang Hwan YEOM, Seoul (KR); Sungrae CHO, Seoul (KR); Sukchan LEE, Suwon-si (KR); Seyeon PARK, Seoul (KR); Yujeong SHIN, Geoje-si (KR)

(21) Appl. No.: 16/505,864

(22) Filed: Jul. 9, 2019

(30) Foreign Application Priority Data
Jul. 9, 2018 (KR) 10-2018-0079608

(51) Int. Cl. G01N 33/50; G01N 33/574; A61K 31/375; A61P 35/00

(52) U.S. Cl. CPC G01N 33/5011; G01N 33/57484; G01N 2800/52; A61P 35/00; A61K 31/375

(57) ABSTRACT

The present invention relates to a method for determining response of cancer patient for vitamin C treatment, and a method for treating a cancer. Specifically, the present invention can predict response for vitamin C treatment by measuring the SVCT-2 expression of cancer cell of cancer patient, and can determine the optimal vitamin-C treatment concentration for each patient, thereby reducing side effects of the patient and alleviating economic and physical pain.

### TECHNICAL FIELD

The present invention provides a method for determining an optimal effective amount of vitamin C capable of exhibiting an anticancer effect without proliferating cancer cell, by predicting the vitamin C absorption level of cancer cell obtained from cancer patient, and a method for predicting response for vitamin C treatment.

### BACKGROUND ART

Vitamin C, known as an antioxidant, acts as a pro-oxidant in cancer cells at a high dose and selectively kills cancer cells. Various studies have found that the anticancer effect of vitamin C inhibits cell proliferation and growth through a reactive oxygen species (ROS) production and hydrogen peroxide-mediated mechanism in an in vitro system.

ROS produced in cancer cells causes cell damage and induces oxidative stress in cancer cells with redox state and metabolism. In addition, the pharmaceutical dosage of vitamin C acting as an oxidation promoter showed an anticancer effect in an in vivo system together with production of ascorbate radicals. Many researchers have conducted studies to understand the mechanism of high-dose vitamin C treatment, and as a result, they have hound that the anticancer mechanism of vitamin C affects cytochrome c release in mitochondria and produces ROS to induce apoptosis.

Historically, vitamin C as a cancer therapeutic agent was first proposed by Linus Pauling and Ewan Cameron in 1976. Previous studies have shown that high-dose vitamin C treatment increases the mean survival time. However, other studies such as mayo clinic have shown that vitamin C treatment has no effect on cancer patients. The debate over this vitamin C cancer treatment began with a clash between clinical results of Linus Pauling's research and mayo clinic research.

To overcome this controversy, a number of studies have been conducted to elucidate the anticancer effect and mechanism of vitamin C. Some studies have found that the expression of vitamin C transporter family 2 (SVCT-2) is a crucial factor in cancer treatment of vitamin C, and have confirmed that a hypoxia-inducible factor-positive cell is sensitive to vitamin C treatment, and in addition, there are other studies for developing a vitamin C cancer therapy in combination with chemotherapeutic agents or other drugs.

Although a number of researches have revealed the anticancer mechanism of vitamin C, and have developed a more effective application of vitamin C cancer treatment, it has not to be found yet the reason why the result of mayo clinic showed a lower survival rate in patient groups treated with vitamin C than the placebo group.

The present inventors have tried to clarify the cause of the conflicting results, in which when treating vitamin C in an SVCT-2 expressing cancer cell line, not only the anticancer effect is shown in some patients while others do not exhibit the anticancer effect, but also the disease is rather worsened, and they have conducted the present study to clarify the criteria for screening cancer patients capable of vitamin C treatment.

### DISCLOSURE

### Technical Problem

One embodiment of the present invention relates to a method determining response of cancer patients for vitamin C treatment comprising (1) measuring the vitamin C absorption level of cancer cells obtained from the cancer patients; (2) classifying the cancer patients into a patient having a low absorption level to the vitamin C and a patient having a high absorption level to the vitamin C, by using the vitamin C absorption level of the cancer cells; and (3) determining whether the cancer patients have cancer deterioration or side effects by vitamin C, when the cancer patients are treated with the vitamin C of which the amount does not produce an amount of reactive oxygen species (ROS) being capable of inducing the cancer cell death effectively.

Another embodiment of the present invention relates to a method for screening of anticancer agent candidates comprising (a) measuring the vitamin C absorption level of cancer cells obtained from cancer patients; (b) treating the vitamin C of which the amount does not produce an amount of reactive oxygen species (ROS) being capable of inducing the cancer cell death to the cancer cell having a low absorption level to the vitamin C, and culturing them to facilitate the proliferation of cancer cells; (c) treating anticancer agent candidates to the cultured cancer cell and culturing them; (d) measuring the expression level of any one or more selected from the group consisting of Cyclin D1, CDK4, c-Myc, Ki-67, and E2F1 in the cancer cell treated with the anticancer agent candidates; and (e) determining the candidate as an anticancer agent, when the marker expression level of the cultured cancer cell which is treated with anticancer agent candidates is decreased compared to a cancer cell which is not treated with the anticancer agent candidates.

Other embodiment of the present invention relates to a cancer treatment method comprising measuring a vitamin C absorption level of cancer cells of a patient having a cancer disease; classifying the patient into a vitamin C insensitive group, when the measured vitamin C absorption is a standard value or less, and classifying the patient into a vitamin C sensitive group, when the measured vitamin C absorption is over the standard value; and administering vitamin C into the patient, wherein the vitamin C insensitive group is a case where the relative protein expression of SVCT-2 to the total protein of cancer cells is 0.04% or less, and wherein 1 mM or more of vitamin C is administered when the patient is the vitamin C insensitive group in the administering vitamin C.

Other embodiment of the present invention relates to a method for predicting an effect of vitamin C on cancer treatment, comprising measuring a vitamin C absorption level of a cancer cell of a patient having a cancer disease.

Other embodiment of the present invention relates to a kit for predicting cancer treatment response of vitamin C, comprising means for measuring expression of sodium-dependent vitamin C transporter 2 (SVCT-2) in a cancer cell.

### Technical Solution

A vitamin C (vitamin C, AA) high-dose therapy produces reactive oxygen species (ROS) and selectively damages cancer cells, thereby showing an anticancer effect. Such an anticancer effect of vitamin C is determined by a transporter of vitamin C, sodium-dependent vitamin C transporter 2 (SVCT-2).

The present inventors have demonstrated that when vitamin-C is treated to a cell line expressing SVCT-2 at a high level with different gradients of concentration (10 μM-2 mM), depending on this, an effective anticancer effect is shown. However, in a cell line expressing SVCT-2 at a low level, when treating a high dose of vitamin C (>1 mM), the anticancer effect was shown, but when treating a low dose of vitamin C (<10 μM), cancer cells were rather proliferated. In other words, the complete opposite conflicting results were shown depending on the concentration of vitamin C to be treated in cancer cells, and this was called the hormetic response.

As confirmed in the following experimental examples, the hormetic response was observed in the SVCT-2 high-expressing cell line treated with an SVCT-based inhibitor, and the hormetic response was not shown for low-dose of vitamin C. In other words, the present inventors have confirmed that the hormetic response occurred in a dose-dependent manner to vitamin C together with the expression level of SVCT-2 of cancer cells.

In addition, the hormetic response was shown in an SVCT-2 expressing cancer cell line producing insufficient ROS at an amount incapable of inducing death of cancer cells due to low vitamin C absorption. Additionally, through molecular analysis, it was confirmed that the expression of Ki-67 and other cancer proliferation markers increased in the hormetic response. Such results show that the anticancer effect of vitamin C is dependent on SVCT-2 expression and show that vitamin C plays two roles in cancer cells.

Previously, some clinical results in studies related to anticancer treatment using vitamin C still remained questionable. Specifically, (1) in the mayo clinic research, when administering a sufficient amount of vitamin C into various cancer patients, the survival rate of some patients administered with high-dose vitamin-C was lower than the placebo group, but the cause of this result remained unclear. (2) the cause of the anticancer activity of vitamin C which may or may not change, when the concentration of vitamin C administered in the body in plasma is reduced, and the concentration of vitamin C consistently remained low in blood for about 4 hours by constant oxidation of vitamin C in a short period of time, has not been revealed yet.

The present inventors have revealed that these questions can be explained by the hormetic response which is a dual dose response in a pharmaceutical concept, and can be explained by a U-shaped curve graph.

The present inventors have assumed that the reason, why these conflicting effects are shown in cancer cells treated with vitamin-C, is a change by hormetic proliferation in a dose-dependent manner of vitamin C together with an SVCT-2 expression level of cells when ROS is insufficiently produced in cells. It was predicted that since the insufficient ROS at a level incapable of inducing death of cancer cells facilitates proliferation of cancer cells through the activity of insulin-like growth factor-1 (IGF-1) and Ras genes, vitamin C insufficiently absorbed in cancer cells could not produce ROS in an amount capable of inducing death of cancer cells and may rather cause proliferation of cancer cells.

The present inventors have investigated the effect of vitamin C on cancer cells, after treating vitamin C to cancer cells with a pharmacokinetic concentration gradient (1 μM˜2 mM). As a result, it was confirmed that SVCT-2 acted as a transporter of vitamin C, and the expression of SVCT-2 in cells and the anticancer effect of vitamin C were proportional. In addition, the present inventors have tried to interpret the result of mayo clinics and the previous clinical result, and have revealed that vitamin C perform two kinds of different functions depending on the absorption level of vitamin C by SVCT-2 expression in cancer cells.

In other words, as the result of the experiment, when vitamin C is treated to cancer cells at a high concentration, it acts as an anticancer agent regardless of the expression level of SVCT-2 of cancer cells, but when vitamin C is treated at a low concentration, the hormetic cancer cell proliferation response has occurred in the SVCT-2 low-expression cell line. Specifically, in the SVCT-2 high-expression cell line, even in case of treating vitamin C at a low concentration, the cell growth inhibition and apoptosis response have occurred. This experimental result means that the vitamin-C treatment acts as an effective chemotherapy in the cancer cell line absorbing vitamin C sufficiently in cancer cells while it rather activates proliferation of cancer in the cancer cell absorbing vitamin C insufficiently.

As confirmed in the following examples and experimental examples, when treating vitamin C to the SVCT-2 low-expression cell line at a low concentration (10 μM), the relative expression of Cyclin D1 which is an important factor affecting proliferation and prognosis of cancer cells increased. Cyclin D1 plays an important role in attracting a transcriptional factor such as E2F1, and inhibits p300 to control transcription. From this, it has been confirmed that Cyclin D1 induced by vitamin C at a low concentration is an important factor in causing the hormetic proliferation response, and it has been confirmed that Cyclin D1 and CDK4 co-localization are important factors inducing cell proliferation (FIG. 9c).

In addition, as the result of cell viability analysis, it has been confirmed that the expression increases of c-Myc, Ki-67 and E2F1 functions as a Cyclin D1-related proliferation marker. c-Myc and Ki-67 bind to DNA to increase the cell proliferation activity.

From this result, it has been confirmed that vitamin C exhibits a characteristic as an effective chemotherapeutic agent in the SVCT-2 high-expression cancer cells and damages cancer cells sufficiently in the SVCT-2-low-expression cancer cells when treating vitamin C at a high dose, but the treatment of vitamin C at an insufficient dose stimulates cyclin-D1-mediated cancer cell proliferation.

In other words, the present inventors have demonstrated that the cancer therapy to treat a high dose of vitamin C is an effective therapy for SVCT-2 high-expression cancer cells, and it has few side effects in patients, and have revealed that not only it has a lower effect in SVCT-2 low-expression cancer patients, but also it may be a risk for cancer patients as it rather facilitates proliferation of cancer. In addition, the present inventors have demonstrated that the method for treating cancer by treating vitamin C requires a sufficient amount of vitamin C at a level incapable of inducing proliferation of cancer cells in SVCT-2 low-expression cell line, and requires careful vitamin C treatment concentration control and treatment. In addition, the present inventors have demonstrated that more effective cancer treatment is possible and the previous controversy over vitamin C cancer treatment can be overcome, by using the cancer treatment by high-dose vitamin C treatment, and SVCT-2 inducible agents or chemotherapeutic agents inducing a synergy effect in combination.

Hereinafter, the present invention will be described in more detail.

One embodiment of the present invention relates to a method for determining response of cancer patients for vitamin C treatment. The method for determining response of cancer patients for vitamin C treatment may comprise (1) measuring the vitamin C absorption level of cancer cells obtained from the cancer patients; (2) classifying the cancer patients into subgroup based on the measured vitamin C absorption level; and (3) determining whether the subgroup of cancer patients have a negative response by vitamin C, when the cancer patients are treated with the vitamin C of which the amount does not produce an amount of reactive oxygen species (ROS) being capable of inducing the cancer cell death effectively. It may further comprise administering 1 mM or more of vitamin C to the cancer patients, when the cancer patients are identified into subject cancer patients having a negative response by vitamin C. The negative response by vitamin C may be no treatment effect, cancer deterioration, or side effects by vitamin C. The cancer patients may be classified into a subgroup having cancer cells with a low absorption level to the vitamin C, and a subgroup having cancer cells with a high absorption level to the vitamin C in the step (2).

According to one embodiment of the present invention, the measuring the vitamin C absorption level of the step (1) may be measuring the relative protein expression of sodium-dependent vitamin C transporter 2 (SVCT-2) to the total protein of cancer cells, but the method is not particularly limited.

According to one specific embodiment, the step (2) may be classifying the cancer patients into a patient having a low absorption level to the vitamin C when the relative protein expression of SVCT-2 to the total protein of the cancer cells is in the range of 0.05(%) or less, 0.0001 to 0.05(%), 0.001 to 0.05(%), 0.01 to 0.05(%), 0.015 to 0.05(%), 0.02 to 0.05(%), 0.04(%) or less, 0.0001 to 0.04(%), 0.001 to 0.04(%), 0.01 to 0.04(%), 0.015 to 0.04(%), or 0.02 to 0.04(%), and may be classifying them into cancer cells having a low absorption level, for example, in case of the SVCT-2 low-expression cancer cell line in which the expression of SVCT-2 is 25 ng or less, 0.1 to 25 ng, 1 to 25 ng, 5 to 25 ng, 10 to 25 ng, 20 ng or less, 0.1 to 20 ng, 1 to 20 ng, 5 to 20 ng, or 10 to 20 ng of the cancer cell total protein 50 μg.

Meanwhile, the step (2) may be classifying the cancer patients into a patient having a high absorption level to the vitamin C when the relative protein expression of SVCT-2 to the total protein of the cancer cells is in the range of 0.04(%) or more, more than 0.04(%), more than 0.04 to 1.0(%), more than 0.04 to 0.1(%), more than 0.04 to 0.08(%), more than 0.04 to 0.076(%), 0.045(%) or more, more than 0.045(%), 0.045 to 1.0(%), 0.045 to 0.1(%), 0.045 to 0.08(%), 0.045 to 0.076(%), 0.05(%) or more, more than 0.05(%), 0.05 to 1.0(%), 0.05 to 0.10(%), 0.05 to 0.9(%), 0.05 to 0.08(%), or 0.05 to 0.076(%), and may be classifying them into cancer cells having a high absorption level, for example, in case of the SVCT-2 low-expression cancer cell line in which the expression of SVCT-2 is 20 ng or more, more than 20 ng, 20 to 50 ng, 20 to 45 ng, 20 to 40 ng, 25 ng or more, more than 25 ng, 25 to 50 ng, 25 to 45 ng, or 25 to 40 ng of the cancer cell total protein 50 μg.

The step (3) may be determining the subgroup having cancer cells with a low absorption level to the vitamin C as a cancer patient having negative response by the vitamin C

The step (3) may be determining the subgroup having cancer cells with a high absorption level to the vitamin C as a cancer patient having no cancer deterioration or side effects by the vitamin C.

The cancer patent having negative response by the vitamin C may be a patient treated with a small amount of vitamin C of which the amount does not produce reactive oxygen species (ROS) at a level at which an effective dose of vitamin C can induce death of cancer cells.

The meaning of the “a small amount of vitamin C” means the amount of vitamin C treatment that produces only insufficient reactive oxygen at a level incapable of reaching that cancer cells finally die by facilitating production of reactive oxygen in cancer cells, when treating sufficient vitamin C to cancer cells.

For example, when the expression of SVCT-2 of cancer cells extracted from cancer patients is lower than 20 ng, or lower than 25 ng of the total protein 50 μg, the amount of vitamin C concentration to be treated less than 2 mM at a level of 300 mL or less, 250 mL or less, 1 mL to 250 mL, 5 mL to 250 mL, or 10 mL to 250 mL may be considered as the small amount.

According to one specific embodiment, the type of the cancer is not particularly limited, but it may be one or more cancers selected from the group consisting of colorectal cancer, breast cancer, ovarian cancer and brain tumor, and it may be preferably colorectal cancer or breast cancer, more preferably colorectal cancer.

According to one embodiment of the present invention, a kit for predicting cancer treatment response of vitamin C, comprising means for measuring expression of sodium-dependent vitamin C transporter 2 (SVCT-2) in cancer cells, may be provided.

The means for measuring expression of SVCT-2 may be one for measuring an expression level of mRNA of SVCT-2 gene or its protein, and methods commonly used in the related art may be used.

Specifically, as the result of measuring the relative protein expression of SVCT-2 to the total protein of cancer cells using the kit, when it is in a range of 0.0001 to 0.05, 0.001 to 0.05, 0.01 to 0.05, 0.02 to 0.05, 0.0001 to 0.04, 0.001 to 0.04, 0.01 to 0.04, or 0.02 to 0.04, cancer patients may be distinguished into a cancer patient having cancer cells having a low absorption level to vitamin C, and it may be predicted that the cancer patient rather has a risk of deterioration of prognosis of cancer or side effects by cancer cell proliferation when treating a small amount of vitamin C.

The meaning of the small amount of vitamin C means the content of vitamin C in a content incapable of producing reactive oxygen species (ROS) at a level capable of inducing death of cancer cells.

On the other hand, as the result of measuring the relative protein expression of SVCT-2 to the total protein of the cancer cells using the kit, when it is in a range of 0.045 to 1.0, preferably 0.05 to 0.10, more preferably 0.05 to 0.08, the most preferably 0.05 to 0.07, cancer patients may be distinguished into a cancer patient having cancer cells with a high absorption level to vitamin C, and it may be predicted that the cancer patient shows the anticancer effect in proportion to the treated concentration of vitamin C and has low possibility of side effects such as cancer cell proliferation and the like.

According to another embodiment of the present invention, provided is a method for screening of anticancer agent comprising (a) measuring the vitamin C absorption level of cancer cells obtained from cancer patients; (b) contacting the cancer cells having a low absorption level to the vitamin C with the vitamin C of which the amount does not produce an amount of reactive oxygen species (ROS) being capable of inducing the cancer cell death, and culturing them to facilitate the proliferation of cancer cells; (c) treating the cultured cancer cells with anticancer agent candidates and culturing them; (d) measuring the expression level of any one or more marker selected from the group consisting of Cyclin D1, CDK4, c-Myc, Ki-67, and E2F1 in the cancer cell treated with the anticancer agent candidates; and (e) determining the candidate as an anticancer agent, when the expression level of the marker in the cultured cancer cell which is treated with anticancer agent candidates is decreased compared to a cultured cancer cell which is not treated with the anticancer agent candidates.

The type of cancer and the step of measuring the absorption level to vitamin C which obtained cancer cells of cancer patients have are the same as described above.

As confirmed in the following examples, in case of the cancer cell determined as having a low absorption level to vitamin C, when treating a small amount of vitamin C, the expression of Cyclin D1, CDK4, c-Myc, Ki-67 and E2F1 that are cancer proliferation factors increased.

Thus, in case that the expression level of any one or more of Cyclin D1, CDK4, c-Myc, Ki-67, and E2F1, preferably all the markers is reduced, when treating an anticancer agent candidate to a cancer cell in which cancer proliferation is facilitated by vitamin C, the candidate may be determined as an anticancer agent.

According to one embodiment of the present invention, a cancer treatment method comprising measuring a vitamin C absorption level of cancer cells of a patient having a cancer disease; classifying the patient into a vitamin C sensitive group, or a vitamin C insensitive group; and administering vitamin C into the patient.

The vitamin C insensitive group is a case where the relative protein expression of SVCT-2 to the total protein of cancer cells may be 0.05% or less, 0.0001 to 0.05%, 0.001 to 0.05%, 0.01 to 0.05%, 0.02 to 0.05%, 0.04% or less, 0.0001 to 0.04%, 0.001 to 0.04%, 0.01 to 0.04%, or 0.02 to 0.04%. The vitamin C sensitive group may be cancer patients who are not classified into the vitamin C insensitive group.

In the step of administering vitamin C, when the patient is the vitamin C insensitive group, 1 mM or more of vitamin C may be administered. In the step of administering vitamin C, when the patient is the vitamin C sensitive group, vitamin C may be administered without dose limitations.

According to one embodiment of the present invention, provided is a method of treating cancer, comprising defining an insensitive subgroup and a sensitive subgroup of cancer patients by measuring a vitamin C absorption level of cancer cells obtained from a cancer patient and classifying the cancer patient into the insensitive subgroup or the sensitive subgroup based on the measured vitamin C absorption level; and administering vitamin C into the cancer patients, wherein the insensitive subgroup shows 0.05% or less of the relative protein expression of SVCT-2 to the total protein of cancer cells in cancer cells obtained from the patient, and wherein 1 mM or more of vitamin C is administered into the cancer patients of insensitive subgroup. The concentration of vitamin C being administered into the cancer patients of insensitive subgroup may be at a concentration such that the concentration of vitamin C reaching cancer cells is 1 mM or more.

The insensitive subgroup may be a group of patients showing a low absorption level to the vitamin C, or low relative protein expression of SVCT-2 to the total protein of cancer cells in cancer cells obtained from the patient, for example 0.05(%) or less of the relative protein expression of SVCT-2 to the total protein of the cancer cells.

The sensitive subgroup may be a group of patients who are not classified as the insensitive subgroup. Specifically, the sensitive subgroup may be a group of patients showing a high absorption level to the vitamin C, or high relative protein expression of SVCT-2 to the total protein of cancer cells in cancer cells obtained from the patient, for example more than 0.05(%) of the relative protein expression of SVCT-2 to the total protein of the cancer cells.

According to one embodiment of the present invention, provided is a method of treating a cancer, comprising administering 1 mM or more of vitamin C to a patient identified as a subject cancer patient having a negative response by vitamin C. The concentration of vitamin C being administered into the cancer patients of insensitive subgroup may be at a concentration such that the concentration of vitamin C reaching cancer cells is 1 mM or more.

According to one embodiment of the present invention, any of a variety of modes of administration may be used. For example, administration may be intravenous, topical, oral, intranasal, subcutaneous, intraperitoneal, intramuscular, intratumor, intradermal, mucosal, intrarectal, intravaginal, inhalation, or aerosol.

The vitamin C of the present invention may be administered to a subject by any of many different routes. For example, the vitamin C may be administered intravenously, intraperitonealy, subcutaneously, intranasally, orally, transdermally, intradermally, intramuscularly, intravaginally, intrarectally, and via aerosol for inhalation delivery. Suitable dosing regimes may be determined by taking into account factors well known in the art including, for example, the age, weight, sex, and medical condition of the subject; the route of administration; the desired effect; and the particular conjugate and formulation employed.

According to one embodiment of the present invention, vitamin C may be administered into a patient of the insensitive subgroup at a concentration such that the concentration of vitamin C reaching cancer cells is 1 mM or more.

### Advantageous Effects

The present invention provides a method for predicting a cancer treatment effect using vitamin-C by measurement and evaluation of expression of SVCT-2 of cancer cell, in cancer treatment using vitamin C as an anticancer agent. With the method, it is possible to determine the optimal effective amount of vitamin-C treatment for application of vitamin-C therapy to cancer patient, and minimize side effects, thereby alleviating the economic and physical pain of the patient.

### BRIEF DESCRIPTION OF DRAWINGS

FIG. 1a shows the result of comparing expression of SVCT-2 in a colorectal cancer cell line by western blot. GAPDH was used as a loading control group.

FIG. 1b shows the result of quantitative analysis of the mass of SVCT-2 compared to each total cell protein 50 ug of HCT116, HCT15 and DLD-1 that are SVCT-2 low-expression cell lines, and Sw480, Sw620 and Lovo cell lines that are high-expression cell lines.

FIG. 1c shows the standard curve of absorbance of the recombinant SVCT2 protein.

FIG. 1d shows the relative SVCT-2 expression of each colorectal cancer cell line by determining by image J program analysis (black bar), and shows the cell viability of the colorectal cancer cell line treated with 1 mM vitamin C (white bar).

FIG. 1e shows the result of SVCT-2 expression HPLC analysis used for investigating the absorption of vitamin C in the colorectal cancer cell line.

FIG. 2a shows the result of measuring the cell viability when treating vitamin C to the SVCT-2 low-expression cell line with a concentration gradient. In the SVCT-2 low-expression cell line, when treating vitamin C with a concentration gradient, the hormetic response occurred.

FIG. 2b shows the result of measuring the cell viability when treating vitamin C to the SVCT high-expression cell line with a concentration gradient. In the SVCT-2 high-expression cell line, the hormetic response did not occur.

FIG. 2c shows the relative expression of p53 of each cell line after vitamin C (AA) no-treatment (white bar), 10 μM treatment (black bar) and 1 mM treatment (grey bar) to each of DLD-1 and HCT15 that are SVCT-2 low-expression cell lines and Sw620 and Sw480 that are SVCT-2 high-expression cell lines.

FIG. 2d shows the relative expression of a cancer cell proliferation confirming factor, Cyclin D1 in each cell line after vitamin C (AA) no-treatment (white bar), 10 μM treatment (black bar) and 1 mM treatment (grey bar) to each of DLD-1 and HCT15 that are SVCT-2 low-expression cell lines and Sw620 and Sw480 that are SVCT-2 high-expression cell lines.

FIG. 2e shows the relative expression of a cancer cell proliferation confirming factor, E2F1 in each cell line after vitamin C (AA) no-treatment (white bar), 10 μM treatment (black bar) and 1 mM treatment (grey bar) to each of DLD-1 and HCT15 that are SVCT-2 low-expression cell lines and Sw620 and Sw480 that are SVCT-2 high-expression cell lines.

FIG. 2f shows the relative expression of a cancer cell proliferation confirming factor, Ki-67 in each cell line after vitamin C (AA) no-treatment (white bar), 10 μM treatment (black bar) and 1 mM treatment (grey bar) to each of DLD-1 and HCT15 that are SVCT-2 low-expression cell lines and Sw620 and Sw480 that are SVCT-2 high-expression cell lines.

FIG. 3a shows the ROS production analysis result in DLD-1 which is an SVCT-2 low-expression cell line, and shows the result of detecting ROS produced in DLD-1 cell line by DCF-Da staining.

FIG. 3b shows the ROS production analysis result in an SVCT-2 low-expression cell line, HCT15, and shows that the ROS produced in HCT15 was detected when treating 1 mM vitamin C, but it was not detected when treating 10 μM.

FIG. 4a shows the expression of a cancer proliferation marker, c-Myc, and the localization of c-Myc in the cell line, after vitamin-C no-treatment/10 μM treatment/1 mM treatment to an SVCT-2 low-expression cell line, DLD-1.

FIG. 4b shows the expression of a cancer proliferation marker, c-Myc, and the localization of c-Myc in the cell line, after vitamin-C no-treatment/10 μM treatment/1 mM treatment to an SVCT-2 low-expression cell line, HCT15.

FIG. 4c shows the result of comparative analysis of Bax, c-Myc and Cyclin D1 expression in a cell by western blot, after vitamin-C no-treatment/10 μM treatment/1 mM treatment to an SVCT-2 low-expression cell line, HCT15. β-actin was used as a loading control group.

FIG. 5a shows the assay result of the cell viability after no treatment/10 μM treatment/1 mM treatment of vitamin C to an SVCT-2 high-expression cell line, Sw480, and the cell viability when treating an SVCT-2 expression inhibitor, phloretin, and vitamin C together to Sw480. It was confirmed that the cell viability was increased than no-treatment and the hormetic response occurred, when treating a small amount (10 μM) of vitamin C to the high-expression cell line treated with the SVCT-2 expression inhibitor.

FIG. 5b shows the assay result of the cell viability after no treatment/10 μM treatment/1 mM treatment of vitamin C to an SVCT-2 high-expression cell line, Sw620, and the cell viability when treating an SVCT-2 expression inhibitor, phloretin, and vitamin C together to Sw480. It was confirmed that the cell viability was increased than no-treatment and the hormetic response occurred, when treating a small amount (10 μM) of vitamin C to the high-expression cell line treated with the SVCT-2 expression inhibitor.

FIG. 5c shows the ROS assay result using DCF-Da staining in each cell line, in case of no treatment/10 μM treatment of vitamin C/1 mM treatment of vitamin C/treatment of 10 μM vitamin C and P (phloretin) to an SVCT-2 high-expression cell line, Sw620.

FIG. 5d shows the ROS assay result using DCF-Da staining in each cell line, in case of no treatment/10 μM treatment of vitamin C/1 mM treatment of vitamin C/treatment of 10 μM vitamin C and P (phloretin) to an SVCT-2 high-expression cell line, Sw480.

FIG. 5e shows the qRT-PCR analysis result of E2F1 and Ki-37 in the Sw620 cell line, after no treatment/treatment of 10 μM vitamin C and phloretin together to an SVCT-2 high-expression cell line, Sw620. The hormetic response occurred in the SVCT-2 high-expression cell line in which the SVCT-2 expression was inhibited by treating phloretin.

FIG. 5f shows the qRT-PCR analysis result of E2F1 and Ki-37 in the Sw480 cell line, after no treatment/treatment of 10 μM vitamin C and phloretin together to an SVCT-2 high-expression cell line, Sw480. The hormetic response occurred in the SVCT-2 high-expression cell line in which the SVCT-2 expression was inhibited by treating phloretin.

FIG. 6a shows the expression of a cancer proliferation marker, c-Myc, and the localization of c-Myc in the cell line, after vitamin-C no-treatment/10 μM treatment/1 mM treatment/simultaneous treatment of vitamin-C 10 μM+phloretin to an SVCT-2 high-expression cell line, Sw620.

FIG. 6b shows the expression of a cancer proliferation marker, c-Myc, and the localization of c-Myc in the cell line, after vitamin-C no-treatment/10 μM treatment/1 mM treatment/simultaneous treatment of vitamin-C 10 μM+phloretin to an SVCT-2 high-expression cell line, Sw480.

FIG. 6c confirms the expression of the cancer proliferation marker in an SVCT-2 high-expression cell line, Sw620, and shows the result of western blot analysis of Bax, c-Myc and Cyclin D1 in the Sw620 cell line, after co-treatment of vitamin C and phloretin. β-actin was used as a loading control group.

FIG. 6d confirms the expression of the cancer proliferation marker in an SVCT-2 high-expression cell line, Sw480, and shows the result of western blot analysis of Bax, c-Myc and Cyclin D1 in the Sw480 cell line, after co-treatment of vitamin C and phloretin. β-actin was used as a loading control group.

FIG. 7 is a schematic diagram showing the cell response mechanism according to the SVCT-2 expression in cancer cells and the concentration of treated vitamin C.

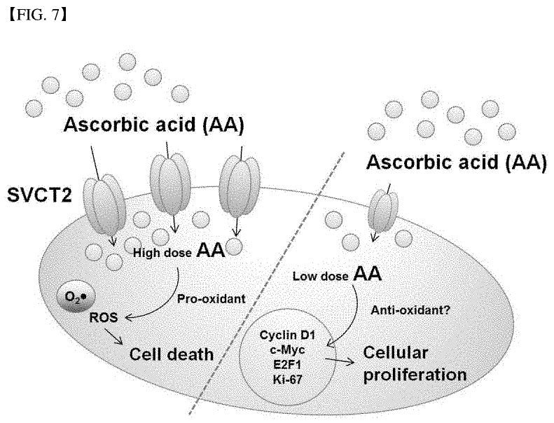

FIG. 8 shows the result of confirming the concentration of phloretin to be treated for excluding cytotoxicity with the cell viability assay.

FIG. 9a is the result of measuring the vitamin C absorption change after vitamin C (AA) no-treatment/1 mM treatment/co-treatment of vitamin C 1 mM+phloretin 20 μM in Sw480 and Sw620 that are SVCT-high-expression cell lines.

FIG. 9b is the result of confirming that the cell proliferation occurs due to binding of Cyclin D1 and CDK4, of which expression increased, when the hormetic response occurred, in an SVCT-2 low-expression cell line, DLD-1, using ICC.

FIG. 9c is the result of confirming that the cell proliferation occurs due to binding of Cyclin D1 and CDK4, of which expression increased, when the hormetic response occurred, in an SVCT-2 low-expression cell line, HCT15 cell. using ICC.

### DETAILED DESCRIPTION OF THE EMBODIMENTS

Hereinafter, the present invention will be described in more detail by the following examples. However, these examples are intended to illustrate the present invention only, but the scope of the present invention is not limited by these examples.

### EXPERIMENTAL EXAMPLE 1

### Cancer Cell Line Culturing and Reagents

Human colorectal cancer cells were cultured in RPMI1640 medium Gilbco, Cergy Pontoise, France) containing 10% bovine fetal serum (PAN Biotech, Aidenbach, Germany) and 1% Penstrep (PAN Biotech) in a humidified incubator comprising 5% CO₂ under the condition of 37° C. Vitamin C was purchased from BCWORLD PHARM (BCWORLD PHARM. CO, Seoul, Korea), and phloretin was purchased from sigma Aldrich (Sigma, St. Louis, Mo., USA).

The human colorectal cancer cell lines used in the experiment were Sw480, Sw620, HCT116, HCT15, SNU-C4, SNU-05, DLD-1, LoVo, and CoLo-205 cell lines commonly used in the related art. All the colorectal cancer cell lines used in the experiment of the present invention were received from Dr. Yu Byung-Chul of National Cancer Center.

### EXPERIMENTAL EXAMPLE 2

### Cell Viability Analysis

The cell viability was measured by Neutral red (sigma) assay. Cells (1×10⁴/each well) were inoculated on a 96-well plate and were cultured for 24 hours and were treated with vitamin C for 4 hours, and then were washed with PBS (Pan Biotech), and were further cultured in RPMI1640 without vitamin C for 20 hours. They were washed twice and were stained.

### EXPERIMENTAL EXAMPLE 3

### qRT-PCR Analysis

According to the method provided by the manufacturer, the total RNA of cells was extracted using TRI reagent (MRC; Molecular Research Center, Cincinnati, Ohio, USA). The RNA concentration was determined using a spectrophotometer reading the absorbance at 260 nm. cDNA was synthesized using mML-V reverse transcriptase (Bioneer Co, Daejeon, Republic of Korea) according to the protocol provided by the manufacturer. Then, using SYBR Premix Ex Taq (TaKaRa, Otsu, Shinga, Japan) and Rotor-Gene Q system (Qiagen, Chadstone, Victoria, Australia), quantitative real-time PCR was carried out. Data were analyzed using Rotor-Gene Q series software version 2.3.1 (Qiagen).

The following genes were amplified using primers shown in the following Table 1:

TABLE 1

| Ampli- | | | |
|---|---|---|---|
| fication | Primer | | SEQ |
| gene | name | Primer sequence (5′ > 3′) | ID NO |
| P53 | P53-F | AGGCCTTGGACCTCAAGGATG | 1 |
| | P53-R | TGAGTCAGGCCCTTCTGTCT | 2 |
| Cyclin D1 | Cyclin D1-F | GCTGCCAAGTGGAAACCARC | 3 |
| | Cyclin D1-R | CCTCCTTCTGCACACATTTGAA | 4 |
| E2F1 | E2F1-F | ATGTTTTCCTGTGCCCTGAG | 5 |
| | E2F1-R | TGGTGGTGGTGACACTATGG | 6 |
| Ki-67 | Ki-67-F | ACGCCTGGTTACTATCAAAAGG | 7 |
| | Ki-67-R | CAGACCCATTTACTTGTGTTGGA | 8 |

### EXPERIMENTAL EXAMPLE 4

### Western Blot

Protein was extracted from frozen tissue using PRO-PREP protein extraction kit according to the instruction of the manufacturer. The protein concentration was measured using Bradford assay (Bio-RAD, Munich, Germany)

After denaturalizing protein 30 μg in sample buffer at 95° C. for 6 minutes, samples were loaded on 12% SDS-polyacrylamide gel and were transferred to a nitrocellulose blotting membrane. Subsequently, the membrane was blocked with 5% skim milk in Tris-buffered saline. Then, after washing it with Tris-buffered saline-0.10% Tween 20 three times, the membrane was cultured at 4° C. overnight with anti-cyclin D1 (1:2500; NB600-584; Novus biologics), anti-c-Myc (1:2500; NB200-108; Novus biologics), anti-Bax (1:2000; 2774; Cell Signaling Technology, Beverly, Mass., USA), and anti-beta-actin (1:5000; ab20272; Abcam, Cambridge, Mass., USA) antibodies. Then, after washing it with Tris-buffered saline-0.10% Tween 20 4 times for 20 minutes, the membrane was cultured with a secondary anti-rabbit, anti-rat or anti-goat antibody for 1 hour. After additional washing, immunoreactive bands exposed to ECL substrate (Pierce, Rockford, Ill., USA) and X-ray film (Agfa-Gevaert N.V, Septestraat, Mortsel, Belgium) were detected.

### EXPERIMENTAL EXAMPLE 5

### Immunocytochemistry

Cells treated with vitamin C were immobilized with 4% paraformaldehyde for 10 minutes and were washed with PBS 3 times for 5 minutes, and then permeable buffer (Biolegend, San Diego, Calif., USA) was treated for 10 minutes. Then, cells were washed with PBS 3 times and were cultured with anti-c-Myc (1:500; NB200-108, Novus biologics) antibody at 4° C. overnight. Then, after washing with PBS 4 times, cells were cultured with a secondary anti-mouse-TRITC (1:1000; ab6786; Abcam) conjugated antibody. The stained cells were observed with a confocal microscope, and the image was treated by zen black edition program.

### EXAMPLE 1

### Measurement of the Absorption Level and Cell Viability of Vitamin C According to SVCT-2 Expression of Cancer Cell Lines

The present inventors measured the SVCT-2 expression, vitamin C absorption and cytotoxic effect of vitamin C in various kinds of colorectal cancer cell lines.

1-1. SVCT-2 Expression in Colorectal Cancer Cell Lines

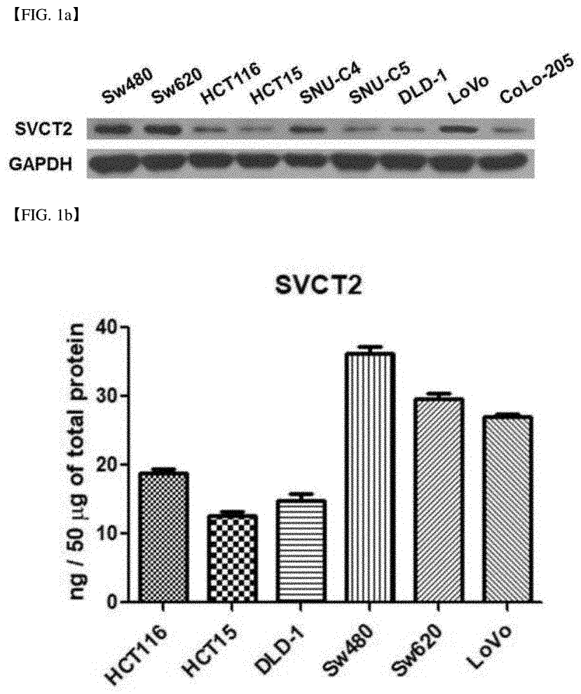

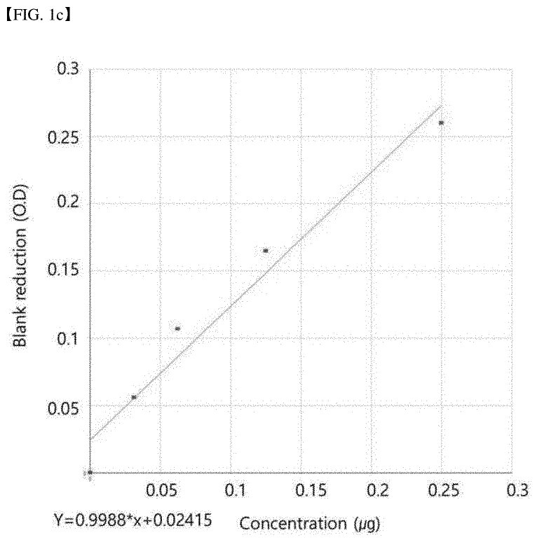

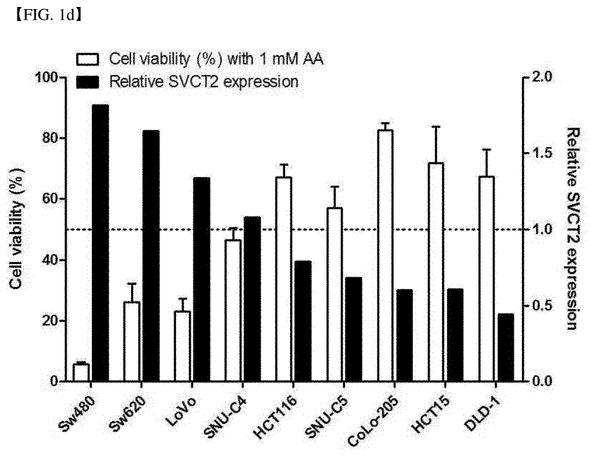

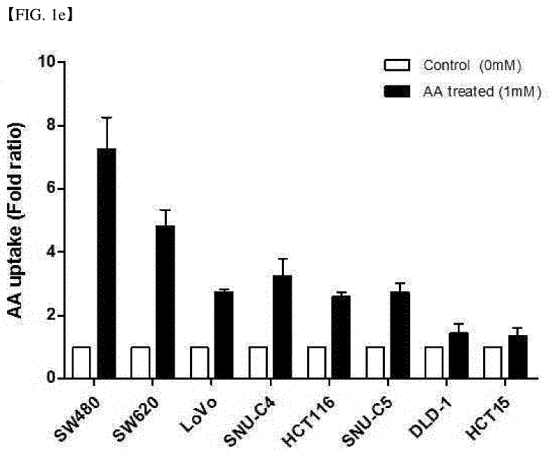

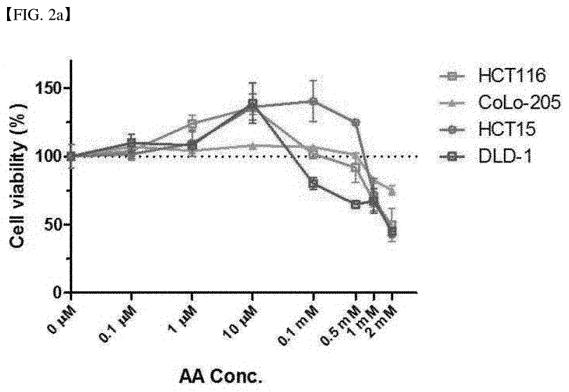

The present inventors analyzed the SVCT-2 expression in colorectal cancer cell lines (Sw480, Sw620, HCT116, HCT15, SNU-C4, SNU-05, DLD-1, LoVo, CoLo-205) using the western blot method of Experimental example 4, and the result was shown in FIG. 1a.

FIG. 1a shows the result of analyzing the SVCT-2 expression in each colorectal cancer cell line with western blot. GAPDH was used as a loading control group. The western blot analysis was conducted by the method of Experimental example 4.

As shown in FIG. 1a, the SVCT-2 expression in each colorectal cancer cell line was shown differently. Specifically, Sw480, Sw620, and Lovo expressed SVCT-2 at a high level, but HCT116, HCT15 and DLD-1 expressed SVCT-2 at a low level.

1-2. Quantitative Analysis of SVCT-2 Expression in Colorectal Cancer Cell Lines

ELISA analysis was carried out in order to quantitatively measure the amount of SVCT-2 to be expressed in each cancer cell line.

Specifically, after quantifying the total protein of cancer cells, the total protein 50 μg and a recombinant SVCT-2 protein (Novousbiologics, H00009962-P01) were under serial dilution, and 0.25 mg, 0.125 mg, 0.0625 mg and 0.03125 mg were put in wells, and after that, they were diluted to 1:200 in carbonate coating buffer (10 mM NaCO₃, 35 mM NaHCO₃, pH 9.6) and then 100 ul each was coated on each well in a 96 well plate. The antigen coating process was progressed at 25° C. for 4 hours. Then, after washing using TBS-T, an anti-SVCT2 antibody (Novous biologics, NBP2-1339) was diluted to 1:500 in PBS per well and 100 ul per well was added, and then it was reacted at 4° C. for 16 hours. After washing using TBS-T, HRP conjugated anti-rabbit igG antibody was diluted in PBS and the concentration was matched to 20 ug/ml, and 100 ul per well was added and it was reacted at 25° C. for 1 hours. Then, after washing using TBS-T, 100 ul of TBM solution was added to each well to progress color reaction, and in 15 minutes, 100 ul of 1M H₂SO₄ was added to stop the color reaction, and the color reaction was confirmed, and the result was shown in FIG. 1b.

FIG. 1b shows the result of quantitative analysis of mass of SVCT-2 compared to the total cell protein 50 ug of each of HCT116, HCT15 and DLD-1 cell lines expressing SVCT-2 at a low level and Sw480, Sw620, and Lovo cell lines expressing SVCT-2 at a high level, as the result of western blot analysis of Example 1-1. FIG. 1c shows the standard curve of absorbance of the recombinant SVCT2 protein.

Specific quantitative data of the graph of FIG. 1b were shown in the following Table 2. In HCT116, HCT15 and DLD-1 cell lines, the expression of SVCT-2 of the total protein 50 μg was shown in a range of 10 ng to 20 ng, and in Sw480, Sw620, and LoVo cell lines, the expression of SVCT-2 of the total protein 50 μg was measured to 25 ng to 40 ng of the total protein 50 μg.

TABLE 2

| | SVCT-2 ng/50 μg of total protein | | | | | |
|---|---|---|---|---|---|---|
| Classification | HCT116 | HCT15 | DLD-1 | Sw480 | Sw620 | LoVo |
| Once | 17.907300 | 13.894460 | 16.904090 | 37.971510 | 30.949040 | 26.936200 |
| Twice | 18.910510 | 11.888040 | 13.894460 | 35.965090 | 29.945830 | 27.939410 |
| 3 times | 19.913720 | 11.888040 | 13.894460 | 34.961880 | 27.939410 | 25.932990 |

1-2. Cell Viability Analysis

The cell viability of each colorectal cancer cell line was measured using the method of Experimental example 2, and the result was shown in FIG. 1b.

The black bar in FIG. 1b shows the relative SVCT-2 expression of each colorectal cancer cell line measured by image J program analysis. The white bar in FIG. 1b shows the result of cell viability measurement of colorectal cancer cell lines treated with 1 mM vitamin C.

The relative SVCT2 expression of cell lines was shown as lower in the order of Sw480, Sw620, LoVo, SNU-C4, HCT116, SNU-05, CoLo-205, HCT15, and DLD-1, and as the result of cell viability analysis, it was confirmed that the cytotoxicity of vitamin C was proportional to SVCT-2 expression.

1-3. Measurement of Vitamin C Absorption of SVCT-2 Expressing Colorectal Cancer Cell Lines

In order to measure the vitamin C absorption of each colorectal cancer cell line, the absorption of vitamin C in cells was analyzed with High Performance Liquid Chromatography (HPLC), and the result was shown in FIG. 1c.

FIG. 1c shows the result of SVCT-2 expression HPLC analysis used for investigating the absorption of vitamin C in each colorectal cancer cell line. As the result of HPLC, the absorption of vitamin C was shown the highest in Sw480 cell line having the highest relative SVCT-2 expression, and the vitamin C absorption was shown lower in DLD-1 and HCT-15 having low relative SVCT-2 expression. In other words, it was confirmed that the absorption of vitamin C was proportional to SVCT-2 expression in colorectal cancer cell lines.

### EXAMPLE 2

### Confirmation of Hormetic Response and Anticancer Effect According to the Treated Concentration of Vitamin C in SVCT-2 Cancer Cell Lines

In order to research a role conducted by vitamin C treated at a high concentration or low concentration to cancer cell lines, after treating vitamin C at various concentrations according to a concentration gradient to each cell line, the cell viability analysis was conducted using the method of Experimental example 2.

2-1. Hormetic Response in SVCT-2 Low-Expression Cell Lines

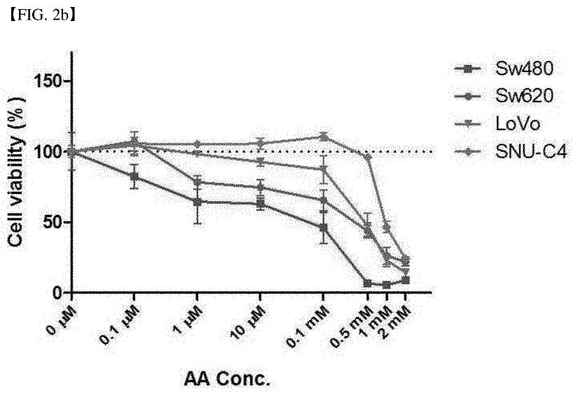

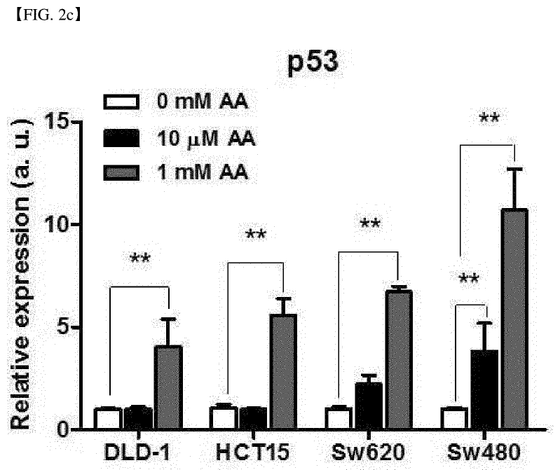

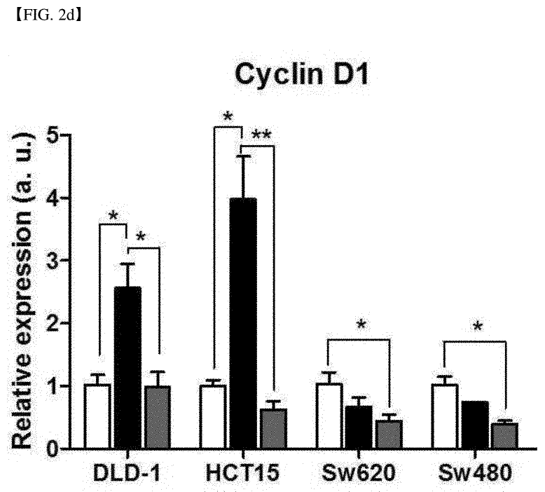

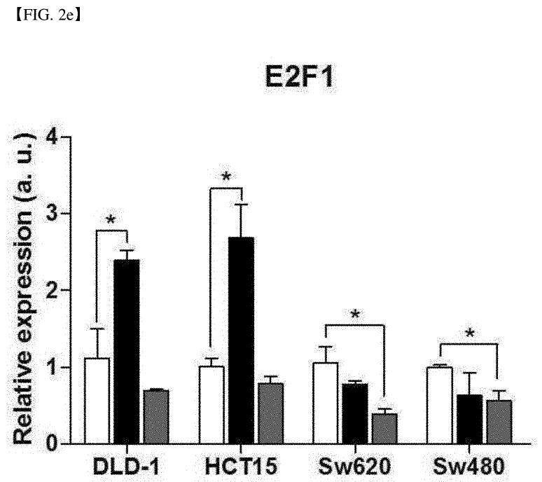

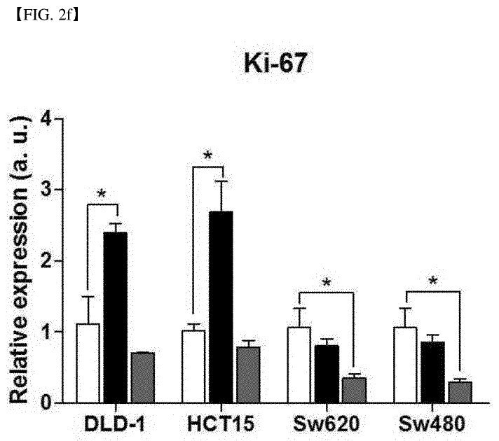

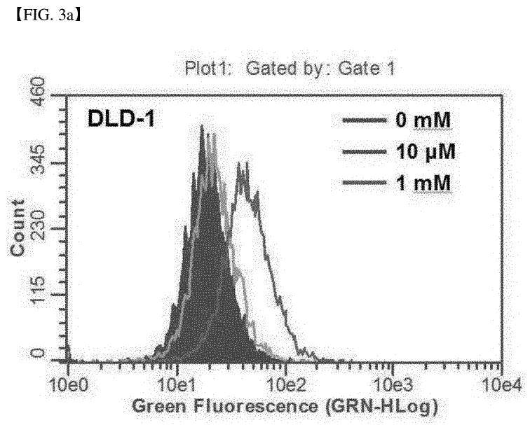

After treating vitamin C to SVCT-2 low-expression cell liens (HCT116, CoLo-205, HCT15, DLD-1) according to a concentration gradient (0 μm to 2 mM), the cell viability was measured, and the result was shown in FIG. 2a.

FIG. 2a shows that the hormetic response occurs when treating vitamin C to SVCT-2 low-expression cell lines according to a concentration gradient. Specifically, when treating vitamin C less than 10 μM, the cell viability increased over 100%, but when treating at a high concentration of 1.0 mM to 2.0 mM or more, the cell viability was dramatically reduced.

In other words, it was confirmed that when treating vitamin C at a high dose (>1 mM), the anticancer effect was shown in SVCT-2 low-expression cell lines, but by contrast, when treating vitamin C at a low dose (<10 μM), the hormetic response occurred and the proliferation of cancer cells were rather induced.

2-2. Hormetic Response in SVCT-2 High-Expression Cell Lines

As same as the SVCT low-expression cell lines, after treating vitamin C to SVCT high-expression cell lines (Sw480, Sw620, LoVo, SNU-C4) according to a concentration gradient (0 μm to 2 mM), the cell viability was measured, and the result was shown in FIG. 2b. It was confirmed that in case of high-expression cell lines, when treating a low concentration of vitamin C, the cell viability was reduced, and as the treated concentration of vitamin C increased, the cell viability was reduced more.

In other words, it was confirmed that the SVCT-2 high-expression cell lines did not show the hormetic response, and all of them showed the anticancer effect when treating high-dose and low-dose vitamin C, and the anticancer effect increased in proportion to the vitamin C treatment concentration.

2-3. qRT-PCR Gene Expression Analysis in SVCT-2 Low-Expression Cell Lines and High-Expression Cell Lines

In order to confirm the apoptosis and hormetic proliferation response by vitamin C, the quantitative real-time polymerase chain reaction (qRT-PCR) gene-expression analysis of p53, Cyclin D1, E2F1 and Ki-67 genes was conducted using the method of Experimental example 3, and the result was shown in FIG. 2c to FIG. 2f.

FIG. 2c shows the relative expression of p53 in each cell line, after vitamin C (AA) no-treatment, 10 μM treatment, and 1 mM treatment to the SVCT-2 low-expression cell lines, DLD-1 and HCT15 and the SVCT-2 high-expression cell lines, Sw620 and Sw480.

As a result, the expression of p53 increased when treating 10 μM and 1 mM ascorbic acid in case of SVCT-2 high-expression cell lines, and they showed apoptosis response related to it, but on the other hand, in SVCT-2 low-expression cell lines, the expression of p53 was induced only in case of treatment of 1 mM vitamin C.

FIG. 2d shows the relative expression of a cancer cell proliferation factor, Cyclin D1 in each cell line, after vitamin C (AA) no-treatment, 10 μM treatment, and 1 mM treatment to the SVCT-2 low-expression cell lines, DLD-1 and HCT15 and the SVCT-2 high-expression cell lines, Sw620 and Sw480.

FIG. 2e shows the relative expression of E2F1 in each cancer cell line using the same experimental method as the preceding experiment.

FIG. 2f also shows the relative expression of Ki-67 in each cancer cell line using the same experimental method.

As shown in FIG. 2d to FIG. 2f, the expression of the cancer cell proliferation confirming factors, Cyclin D1, E2F1 and Ki-67 increased in SVCT-2 low-expression cell line treated with 10 μM vitamin C

In other words, when treating a low concentration of vitamin C to SVCT-2 low-expression cell lines, the hormetic response was induced, and specifically, the expression of cyclin D1, E2F1 and Ki-67 in SVCT-2 low-expression cell lines, DLD-1 and HCT15 increased. On the other hand, in the SVCT-2 high-expression cell lines, Sw620 and Sw480, when treating vitamin C 10 μM or 1 mM, the expression of cyclin D1, E2F1 and Ki-67 was reduced.

The above results show that vitamin C induces an anticancer effect at any concentration in SVCT-2 high-expression cell lines, but in SVCT-2 low-expression cell lines, it induces the anticancer effect as the result of molecular analysis, only when a high concentration of vitamin C is treated, but it induced cell proliferation in a low concentration of vitamin C

### EXAMPLE 3

### ROS Production Analysis in SVCT-2 Low-Expression Cell Lines

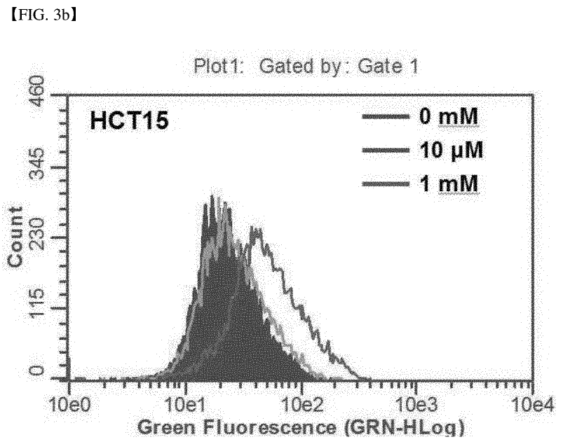

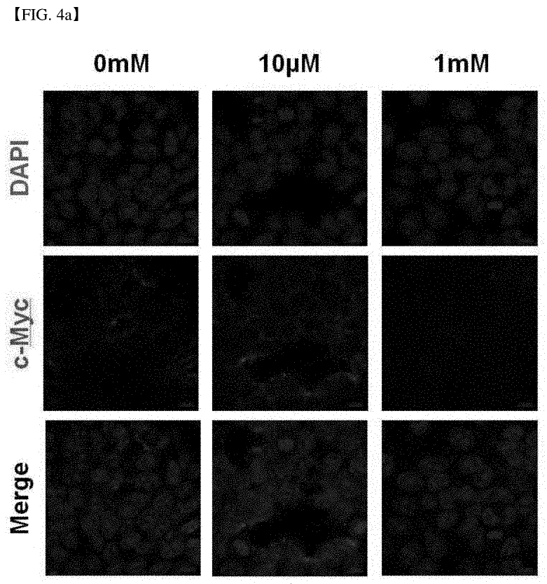

In order to investigate ROS production, when treating vitamin C of 10 μM or 1 mM to SVCT-2 low-expression cell lines (DLD1 and HCT15), after treating each dose of vitamin C to SVCT-2 low-expression cell lines, they were stained with DCF-Da, and the ROS measurement result was shown in FIG. 3a and FIG. 3b.

Specifically, after treating vitamin C to cancer cells for 15 minutes, cells were collected and cells were cultured in PBS solution containing 20 μMd DCF-DA for 10 minutes, and after a washing process with PBS, they were analyzed by FACs.

FIG. 3a shows the result of detecting ROS production in DLD-1 cell line with DCF-Da staining, and FIG. 3b shows the result of detecting ROS production in HCT15 cell line with DCF-Da staining.

In the SVCT-2 low-expression cell lines, DLD-1 and HCT15, ROS was produced only in case of treating 1 mM vitamin C, and it was not produced in case of treating 10 μM. In other words, it was confirmed that when treating a high concentration 1 mM of vitamin C to SVCT-2 low-expression cell lines, the anticancer effect was induced, but when treating a low concentration 10 μM of vitamin C, the anticancer effect was not induced and rather the proliferation of cancer cells was facilitated.

### EXAMPLE 4

### Molecular Analysis of Hormetic Response Occurring When Treating Vitamin C to Cancer Cell Lines

4-1. Measurement of Apoptosis and Proliferation in SVCT-2 Low-Expression Cancer Cell Lines Treated With Vitamin C

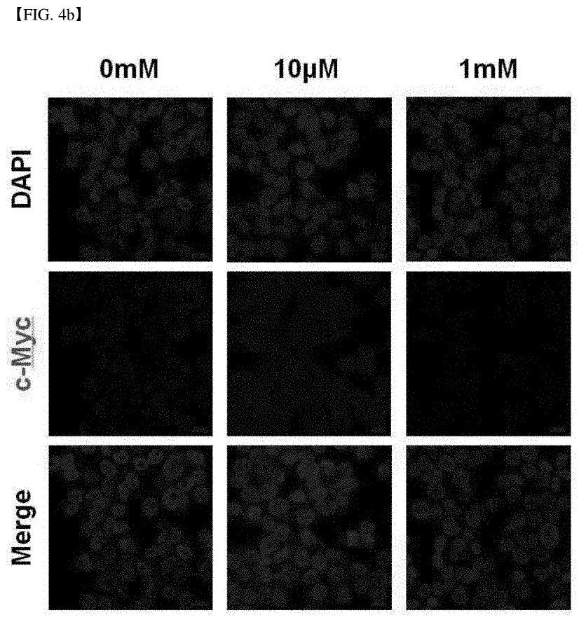

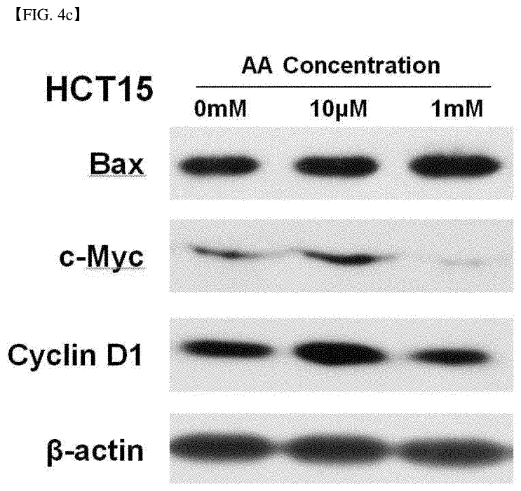

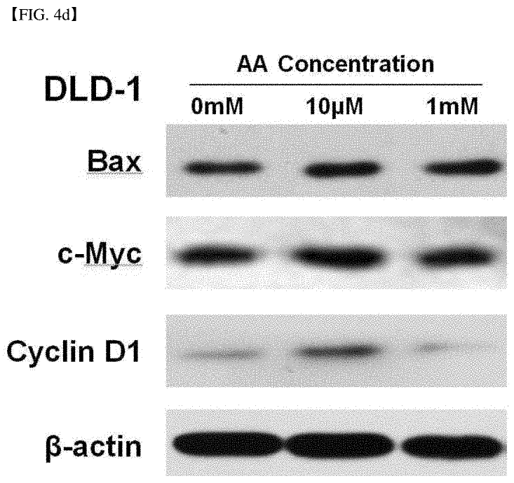

In order to investigate the apoptosis and proliferation of cancer cells by vitamin C treatment in the protein expression level, a low concentration or high concentration of vitamin C was treated to SVCT-2 low-expression cell lines and the western blot analysis of Bax, c-Myc, and Cyclin D1 was conducted using the method of Experimental example 4, and the result was shown in FIG. 4c and FIG. 4d.

FIG. 4c and FIG. 4d shows the result of measuring and analyzing Bax, c-Myc, and Cyclin D1 with western blot after treating vitamin C to SVCT-2 low-expression cell lines, HCT15 and DLD-2. β-actin was used as a loading control group.

In HCT15 and DLD-1, the expression of cancer cell proliferation factors, c-Myc and cyclin D1 increased when treating a low concentration of 10 μM vitamin C, but the expression of the factors was reduced when treating a high concentration of 1 mM vitamin C. However, Bax expression did not significantly change in both vitamin C 1 mM and 10 μM.

4-2. Confirmation of c-Myc Localization

In order to investigate the expression of cancer proliferation markers in SVCT-2 low-expression cell lines and localization where c-Myc is present in cancer cell lines, after treating vitamin C of 1 mM or 10 μM to each SVCT-2 low-expression cell line, HCT15 and DLD-1 cell lines, the immunocytochemistry of Experimental example 5 was conducted, and the result was shown in FIGS. 4a and 4b.

FIG. 4a shows the c-Myc localization in the HCT15 cell line treated with vitamin C with immunocytochemistry, and FIG. 4b shows the c-Myc localization in the DLD-1 cell line treated with vitamin C with immunocytochemistry. As shown in FIGS. 4a and 4b, it was confirmed that c-Myc was hardly localized in a nucleus, and increased c-Myc was almost localized in cytoplasm.

### EXAMPLE 5

### Hormetic Response of SVCT High-Expression Cell Lines Treated With SVCT-2 Family Inhibitor

5-1. Vitamin C Absorption Change Measurement

In order to confirm whether the hormetic proliferation response is medicated from absorption of vitamin C through SVCT-2, the present inventors treated vitamin C 1 mM or vitamin C 10 μM, and an SVCT family inhibitor, phloretin together to SVCT-2 high-expression cell lines.

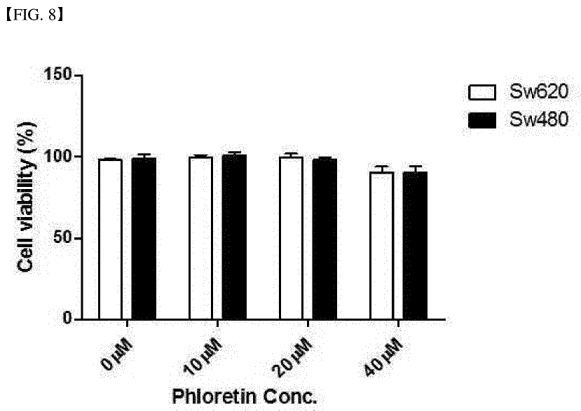

At first, the concentration of phloretin to be treated to exclude cytotoxicity was determined by cell viability assay, and the result was shown in FIG. 8. From this, it was confirmed that the concentration of phloretin used in the present experiment had no cell self-toxicity.

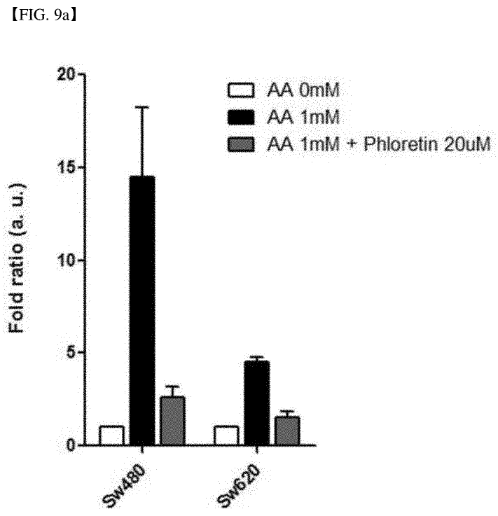

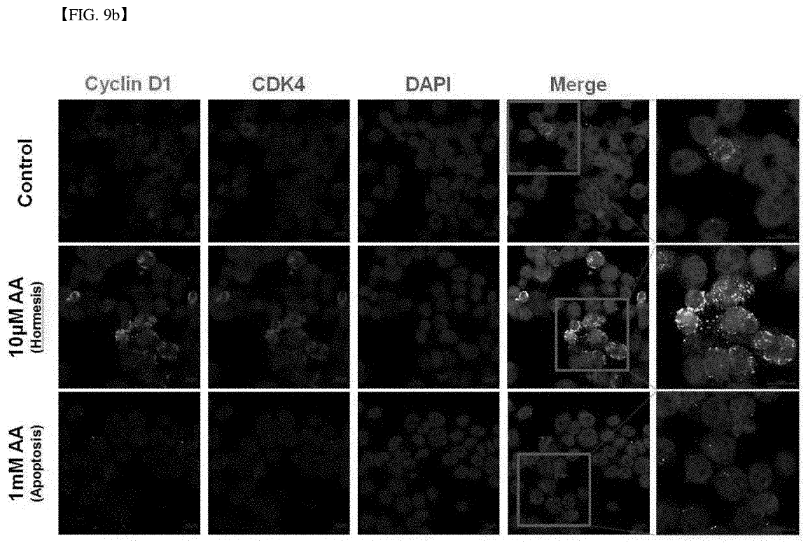

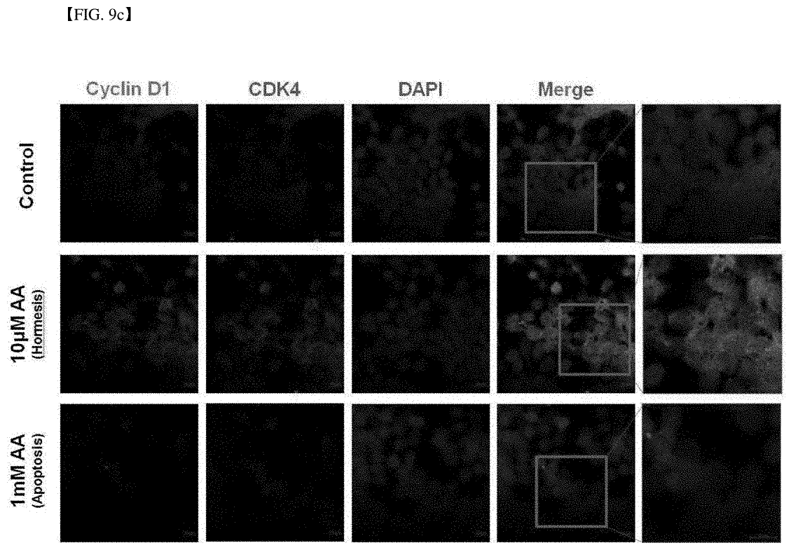

Then, after treating 1 mM vitamin C, or treating vitamin C 1 mM and phloretin 20 μM together to each cell line of the SVCT-2 high-expression cell lines, Sw480 and Sw620, the vitamin C absorption was measured, and the result was shown in FIG. 9a.

FIG. 9a shows the amount of vitamin C in each cell line, and from this, it was confirmed that the intracellular capacity of vitamin C was reduced, when treating phloretin. From this, it was confirmed that the vitamin C absorption of cancer cell lines occurred by mediating SVCT-2.

5-2. Confirmation of Hormetic Proliferation Response in SVCT-2 High-Expression Cell Lines

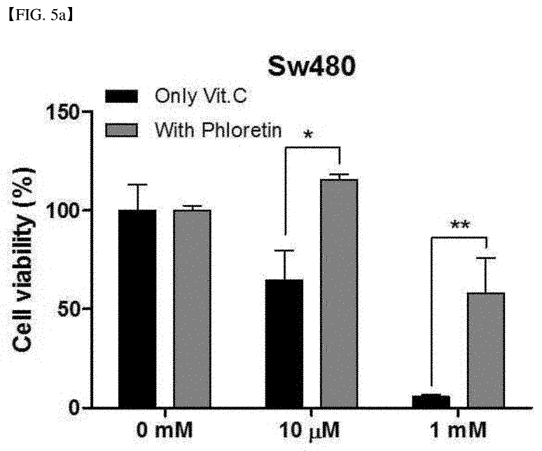

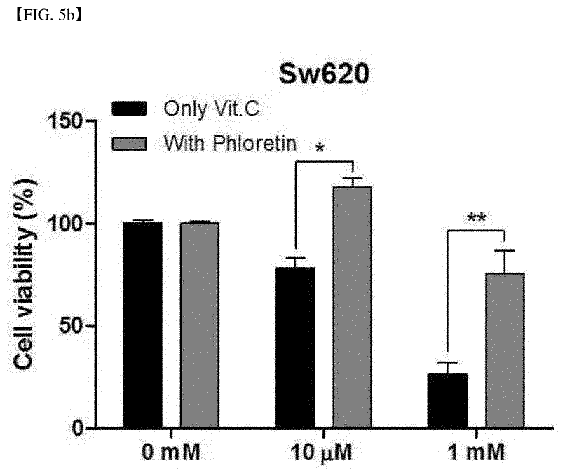

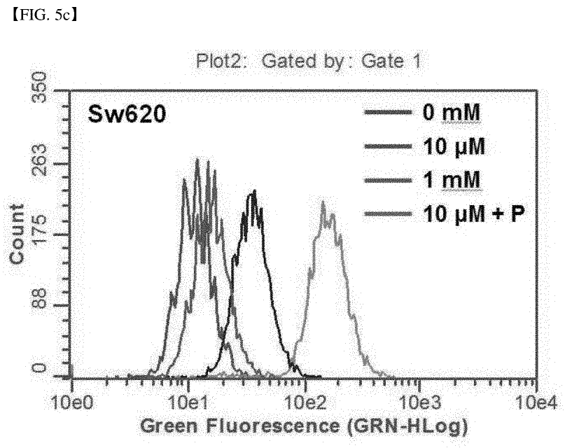

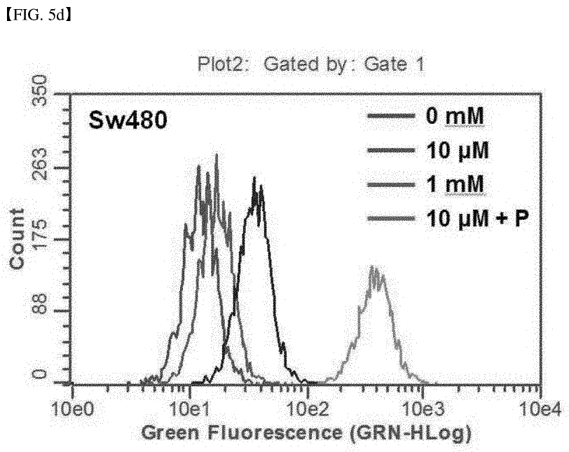

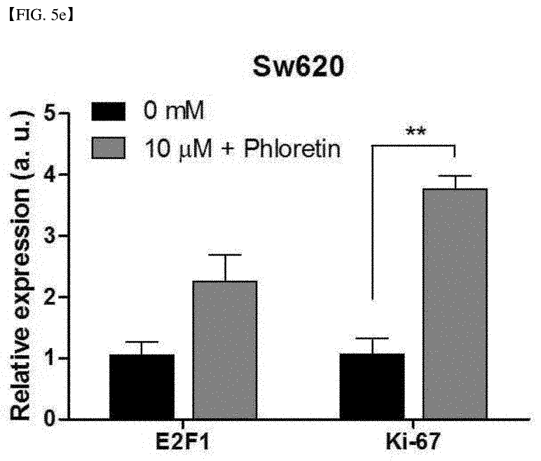

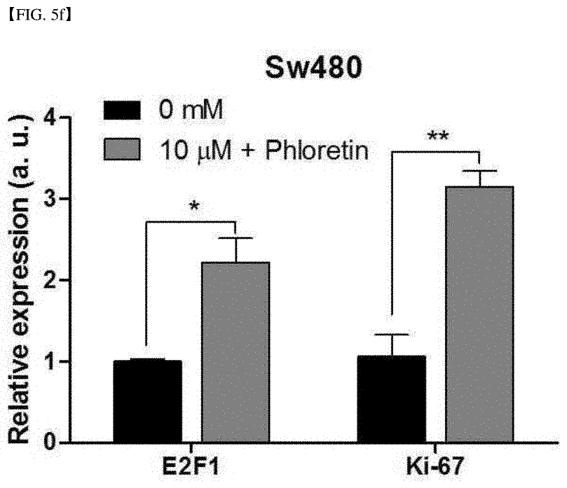

After treating vitamin C of 10 μM or 1 mM, or treating vitamin C and phloretin together, to the SVCT-2 high-expression cell lines, Sw480 and Sw620, the cell viability of each cell line was measured, and the result was shown in FIG. 5a and FIG. 5b.

FIG. 5a shows the result of cell viability measurement measured in Sw620 cell line. As shown in FIG. 5 and FIG. 5b, it was confirmed that in the SVCT-2 high-expression cell lines, the apoptosis response was induced in case of both treating vitamin C 1 mM and treating 10 μM, while the hormetic proliferation response occurred in each cell line of Sw620 and Sw480, when treating an SVCT family inhibitor, phloretin together with vitamin C 10 μM treatment to inhibit the SVCT-2 expression.

In other words, it was confirmed that the SVCT-2 expression inhibition in SVCT-2 high-expression cell lines by phloretin treatment induced the hormetic proliferation when treating a low concentration (10 μM) of vitamin C.

### EXAMPLE 6

### ROS Production by Inhibition of Expression of SVCT-2 in SVCT-2 High-Expression Cell Lines

In order to confirm ROS production in SVCT-2 high-expression cell lines in the apoptosis response and hormetic response, and more specifically confirm the cause of the result of Example 5, the present inventors investigated ROS production using DCF-Da staining in Sw620 cell line and Sw480 cell line, and the result was shown in FIG. 5c and FIG. 5d.

Specifically, after treating vitamin C to cancer cells for 15 minutes, cells were collected and cells were cultured in PBS solution containing 20 μMd DCF-DA for 10 minutes, and after a washing process with PBS, they were analyzed with FACs.

FIG. 5c and FIG. 5d show the ROS analysis result by DCF-Da staining, after treating 10 μM (low concentration) or 1 mM (high concentration) of vitamin C, or 10 μM (low concentration) vitamin C and 20 μM phloretin to Sw620 and Sw480 cell lines. FIG. 5c shows the result of cell lines of Sw620 and FIG. 5d shows the result of cell lines of Sw480.

In other words, when treating 1 mM vitamin C or treating 10 μM vitamin C, ROS was produced respectively in SVCT-2 high-expression cell lines, Sw620 cell line and Sw480 cell line, but when inhibiting the expression of SVCT-2 by treating phloretin, ROS was not sufficiently produced.

### EXAMPLE 7

### Localization of c-Myc and Expression of Cancer Cell Proliferation Markers in SVCT-2 High-Expression Cell Lines

In order to analyze the hormetic proliferation response induced by SVCT-2 inhibited by phloretin treatment in Example 5, the expression of cancer proliferation markers and BAX expression were analyzed using the qRT-PCR of Experimental example 3 and western blot method of Experimental example 4.

7-1. qRT-PCR Analysis of Cancer Proliferation Markers and BAX Expression

After treating vitamin C 10 μM and phloretin to SVCT-2 high-expression cell lines, Sw480 and Sw620 at the same time, the relative expression of Ki-67 and E2F1 used as cancer cell proliferation markers was measured using the qRT-PCR of Example 1, and the result was shown in FIG. 5e and FIG. 5f. FIG. 5e shows the result measured in Sw620 cell line and FIG. 5f shows the result measured in Sw480 cell line.

According to the above result, it could be confirmed that when treating vitamin C 10 μM and phloretin to Sw620 and Sw480 cell lines at the same time, the expression of Ki-67 and E2F1 increased, and that is, cancer cells were proliferated.

7-2. Localization of c-Myc in SVCT-2 High-Expression Cell Lines Under the Hormetic Response Conditions

In order to confirm the cell proliferation by the hormetic response in cancer cell lines, the cell proliferation confirmation experiment through c-Myc was conducted.

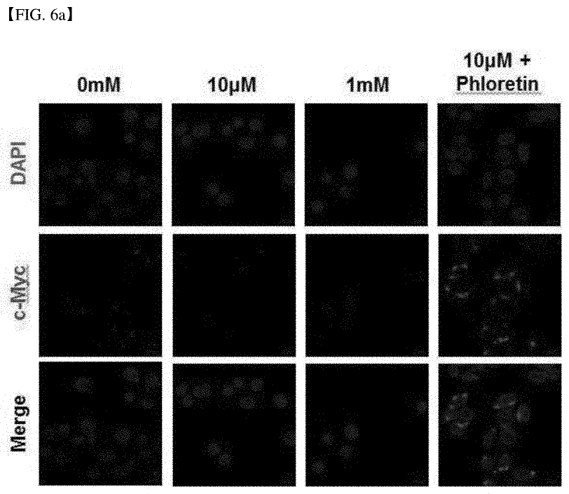

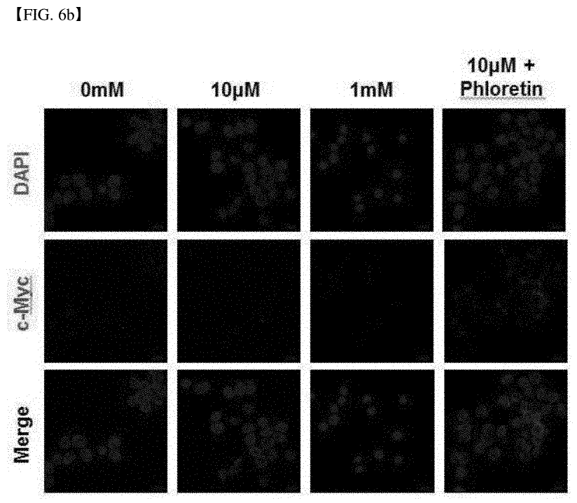

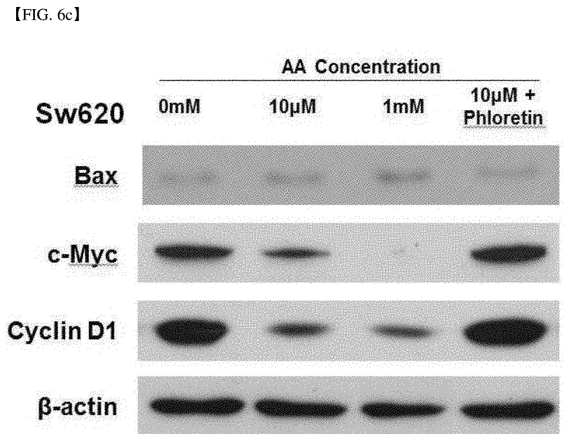

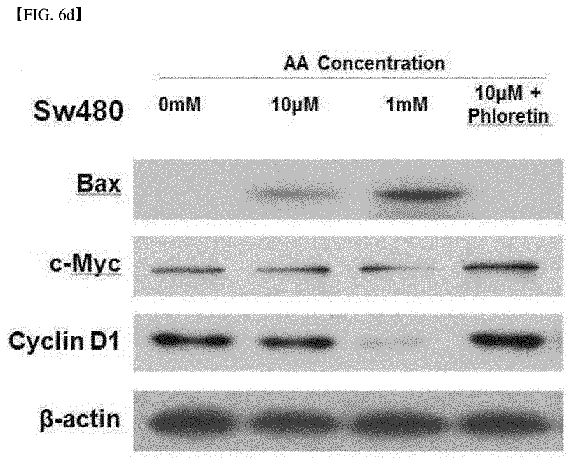

Specifically, after smearing cells on cover glass and culturing them, vitamin C was treated and after washing with PBS, an antigen with Cyclin D1 and CDK4 as antigens was attached to cells, and it was confirmed through fluorescence. The experimental result was shown in FIG. 6a and FIG. 6b.

As could be seen from the above result, the localization of c-Myc in SVCT-2 high-expression cells in which the expression of SVCT-2 was inhibited by treating phloretin together was present at the same location as c-Myc of SVCT-2 low-expression cell lines. In other words, in SVCT-2 high-expression cell lines, the increased localization of c-Myc was present in cytoplasm not a nucleus. From this, it could be seen that the cancer cell proliferation occurred actively when the hormetic response occurred.

7-3. Western Blot of C-Myc and Cyclin D1

After treating vitamin C 10 μM and phloretin to SVCT-2 high-expression cell lines, Sw480 and Sw620 at the same time, the western blot analysis of Bax, c-Myc and Cyclin D1 in cells was conducted, and the result was shown in FIG. 6c and FIG. 6d. FIG. 6c shows the result of Sw620 cell line and FIG. 6d shows the result of Sw480 cell line, and β-actin was used as a loading control group.

As shown in FIG. 6c and FIG. 6d, as the result of western blot, the expression of C-Myc and cyclin D1 increased under the hormetic proliferation condition of treating 10 μM vitamin C and phloretin at the same time, and it was significantly reduced in case of 10 μM vitamin C treatment or 1 mM vitamin C treatment without phloretin treatment. However, the Bax expression increased when treating vitamin C, but the Bax expression by phloretin treatment did not change remarkably.

From the results of FIG. 6c and FIG. 6d, the cancer cell death marker, Bax increased significantly when treating 10 μM vitamin C or 1 mM vitamin C, but there was no particular change when inhibiting intracellular reception of vitamin C with phloretin. The expression of cancer cell proliferation markers, C-Myc and cyclin D1 increased in case of treatment of 10 μM vitamin C and phloretin at the same time. Thus, it could be confirmed that even in a high SVCT2 expression cell line, the hormetic proliferation occurred, when the intracellular reception of vitamin C was not no more than a certain amount.

### EXAMPLE 8

### Cell Viability Analysis According to Concentration-Dependent Expression Inhibition in SVCT-2 High-Expression Cell Lines

After treating 10 μM vitamin C and various concentrations (0 μM, 10 μM, 20 μM or 40 μM) of phloretin to SVCT-2 high-expression cell lines, Sw480 and Sw620 at the same time, the cell viability was measured, and the result was shown in FIG. 8.

FIG. 8 shows the result of cell viability analysis with concentrations of phloretin to be treated to exclude the cytotoxicity, and it was confirmed that phloretin inhibited the intracellular reception of vitamin C, when a concentration having no effect on the cell viability was treated to cancer cell lines.

### EXAMPLE 9

### Analysis of Vitamin C Absorption Change in SVCT-2 High-Expression Cell Lines

9-1: Analysis of Vitamin C Absorption of SVCT-2 High-Expression Cell Lines

The experimental results measured according to the method for measuring the vitamin C absorption of SVCT-2 expression cell lines of Example 1-3, after treating various concentrations (0 mM or 1 mM) of vitamin C and treating 1 mM vitamin C and 20 μM phloretin, for SVCT-2 high-expression cell lines, Sw480 and Sw620 were shown in FIG. 9a.

FIG. 9a shows the result of measuring the vitamin C absorption change when treating vitamin C and the SVCT-2 inhibitor, phloretin, in SVCT high-expression cell lines.

9-2: Confirmation of Localization of c-Myc of SVCT-2 Cell Lines

In order to confirm the expression of cancer proliferation markers, after treating vitamin C in SVCT-2 low-expression cell lines, the immunocytochemistry was conducted to confirm that, and the result was shown in FIG. 9b.

FIG. 9b shows the localization of Cyclin D1 and CDK4 in DLD-1 cell line treated with vitamin C with immunocytochemistry, and from this, it was confirmed that Cyclin D1 and CDK4 co-localization might be an important factor for cell proliferation.

FIG. 9c shows the localization of Cyclin D1 and CDK4 in HCT15 cell line treated with vitamin C with immunocytochemistry, and it was confirmed that Cyclin D1 with increased expression when the hormetic proliferation occurred bound to CDK4 to cause cell proliferation.

1. A method for determining response of cancer patients for vitamin C treatment comprising,
(1) measuring the vitamin C absorption level of cancer cells obtained from the cancer patients;
(2) classifying the cancer patients into subgroup based on the measured vitamin C absorption level; and
(3) determining whether the subgroup of cancer patients have a negative response by vitamin C, when the cancer patients are treated with vitamin C of which the amount does not produce an amount of reactive oxygen species (ROS) being capable of inducing the cancer cell death effectively.

2. The method according to claim 1, wherein the cancer patients are classified into a subgroup having cancer cells with a low absorption level to the vitamin C, and a subgroup having cancer cells with a high absorption level to the vitamin C in the step (2).

3. The method according to claim 1, wherein the step (1) of measuring the vitamin C absorption level is measuring the relative protein expression of sodium-dependent vitamin C transporter 2 (SVCT-2) to the total protein of cancer cells.

4. The method according to claim 2, wherein the step (2) is classifying the cancer patients into a patient having cancer cells with a low absorption level to the vitamin C when the relative protein expression of SVCT-2 to the total protein of the cancer cells is 0.0001 to 0.04(%), and a patient having cancer cells with a high absorption level to the vitamin C when the relative protein expression of SVCT-2 is more than 0.04 to 1.0 (%).

5. The method according to claim 2, wherein the step (2) is classifying the cancer patients into a patient having cancer cells with a low absorption level to the vitamin C when the relative protein expression of SVCT-2 to the total protein of the cancer cells is 0.05 (%) or less, and a patient having cancer cells with a high absorption level when the relative protein expression of SVCT-2 is more than 0.05 (%).

6. The method according to claim 2, wherein in the step (3), the subgroup having cancer cells with a low absorption level to the vitamin C is determined as the cancer patient having a negative response by the vitamin C, when the cancer patients are treated with vitamin C of which the amount does not produce an amount of reactive oxygen species (ROS) being capable of inducing the cancer cell death effectively.

7. The method according to claim 2, wherein in the step (3), the subgroup having cancer cells with a high absorption level to the vitamin C is determined as the cancer patient having no negative response by the vitamin C, when the cancer patients are treated with vitamin C of which the amount does not produce an amount of reactive oxygen species (ROS) being capable of inducing the cancer cell death effectively.

8. The method according to claim 1, wherein the cancer is one or more selected from the group consisting of colorectal cancer, breast cancer, ovarian cancer and brain tumor.

9. A method for screening of anticancer agent comprising
(a) measuring the vitamin C absorption level of cancer cells obtained from cancer patients;
(b) contacting the cancer cells having a low absorption level to the vitamin C with the vitamin C of which the amount does not produce an amount of reactive oxygen species (ROS) being capable of inducing the cancer cell death, and culturing them to facilitate the proliferation of the cancer cells;
(c) treating the cultured cancer cells with anticancer agent candidates and culturing them;
(d) measuring the expression level of one or more marker selected from the group consisting of Cyclin D1, CDK4, c-Myc, Ki-67, and E2F1 in the cultured cancer cell treated with the anticancer agent candidates; and
(e) determining the candidate as an anticancer agent, when the expression level of the marker in the cultured cancer cell which is treated with anticancer agent candidates is decreased compared to a cultured cancer cell which is not treated with the anticancer agent candidates.

10. The method according to claim 9, wherein the cancer is one or more selected from the group consisting of colorectal cancer, breast cancer, ovarian cancer and brain tumor.

11. The method according to claim 9, wherein the step (a) of measuring the vitamin C absorption level is measuring the relative protein expression of sodium-dependent vitamin C transporter 2 (SVCT-2) to the total protein of cancer cells.

12. The method according to claim 9, wherein the cancer cell having a low absorption level to the vitamin C shows 0.0001 to 0.04(%) of the relative protein expression of SVCT-2 to the total protein of the cancer cell.

13. A method of treating a cancer, comprising
defining an insensitive subgroup and a sensitive subgroup of cancer patients by measuring a vitamin C absorption level of cancer cells obtained from a cancer patient and classifying the cancer patient into the insensitive subgroup or the sensitive subgroup based on the measured vitamin C absorption level; and
administering vitamin C into the cancer patients,
wherein the insensitive subgroup shows 0.05% or less of the relative protein expression of SVCT-2 to the total protein of cancer cells in cancer cells obtained from the patient, and
wherein 1 mM or more of vitamin C is administered into the cancer patients of insensitive subgroup.

14. The method according to claim 13, wherein the insensitive subgroup shows 0.04% or less of the relative protein expression of SVCT-2 to the total protein of cancer cells in cancer cells obtained from the patient.

15. The method according to claim 13, wherein the vitamin C is administered topically, intratumorally, mucosally, intravenously, intraperitonealy, subcutaneously, intranasally, orally, transdermally, intradermally, intramuscularly, intravaginally, or intrarectally into the cancer patients.

16. The method according to claim 13, wherein the vitamin C is administered to the insensitive subgroup at a concentration such that the concentration of vitamin C reaching cancer cells is 1 mM or more.

17. The method according to claim 1, wherein further comprising administering 1 mM or more of vitamin C into the subgroup of cancer patients having a negative response by vitamin C.


SEQUENCE LISTING (8 sequences)

SEQ ID NO: 1 - DNA, 21 bp, Artificial Sequence
  (synthetic) P53-F
aggccttggacctcaaggatg

SEQ ID NO: 2 - DNA, 20 bp, Artificial Sequence
  (synthetic) P53-R
tgagtcaggcccttctgtct

SEQ ID NO: 3 - DNA, 20 bp, Artificial Sequence
  (synthetic) Cyclin D1-F
gctgccaagtggaaaccarc

SEQ ID NO: 4 - DNA, 22 bp, Artificial Sequence
  (synthetic) Cyclin D1-R
cctccttctgcacacatttgaa

SEQ ID NO: 5 - DNA, 20 bp, Artificial Sequence
  (synthetic) E2F1-F
atgttttcctgtgccctgag

SEQ ID NO: 6 - DNA, 20 bp, Artificial Sequence
  (synthetic) E2F1-R
tggtggtggtgacactatgg

SEQ ID NO: 7 - DNA, 22 bp, Artificial Sequence
  (synthetic) Ki-67-F
acgcctggttactatcaaaagg

SEQ ID NO: 8 - DNA, 23 bp, Artificial Sequence
  (synthetic) Ki-67-R
cagacccatttacttgtgttgga